SEQUENCE LISTING (1 sequence)

SEQ ID NO: 1 - protein, 263 aa, synthetic construct
  REGION 1..262: note = Sequence of the antigenic protein of Proteasome subunit alpha type 1
MFRNQYDNDVTVWSPQGRIHQIEYAMEAVKQGSATVGLKSKTHAVLVALKRAQSELAAHQKKILHVDNHIGISIAGLTADARLLCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

(19) United States
(12) Patent Application Publication
(10) Pub. No.: US 2023/0333115 A1
(43) Pub. Date: Oct. 19, 2023

(54) KIT FOR DETECTING ANTI-PROTEASOME SUBUNIT ALPHA TYPE 1-IMMUNOGLOBULIN G (IgG) ANTIBODY

(71) Applicant: ZHEJIANG UNIVERSITY, Hangzhou City (CN)

(72) Inventors: Qing YE, Hangzhou City (CN); Jianhua MAO, Hangzhou City (CN); Dandan TIAN, Hangzhou City (CN)

(73) Assignee: ZHEJIANG UNIVERSITY, Hangzhou City (CN)

(21) Appl. No.: 17/856,033

(22) Filed: Jul. 1, 2022

(30) Foreign Application Priority Data
Jul. 1, 2021 (CN) 202110743551.7

(51) Int. Cl. G01N 33/68; G01N 33/543

(52) U.S. Cl. CPC G01N 33/6854; G01N 33/54326; G01N 2800/347; G01N 2333/96425

(57) ABSTRACT

The present disclosure provides a kit for detecting an anti-proteasome subunit alpha type 1-immunoglobulin G (IgG) antibody, including an antigen protein proteasome subunit alpha type 1, a solid phase carrier, a labeled antibody, an antigen diluent, a sample dilution buffer, an antibody diluent, a substrate color development reagent, a washing solution, a standard, a positive quality control, and a negative quality control. In the present disclosure, the kit is capable of detecting the anti-proteasome subunit alpha type 1-IgG antibody in serum to be tested by indirect reaction combined with magnetic particle-based chemiluminescence immunoassay. Autoantibodies against the target antigen protein proteasome subunit alpha type 1 are identified in the serum of a patient with autoimmune nephrotic syndrome for the first time.

### CROSS REFERENCE TO RELATED APPLICATION

This application claims the benefit and priority of Chinese Patent Application No. 202110743551.7, filed on Jul. 1, 2021, the entire disclosure of which is hereby expressly incorporated by reference.

### TECHNICAL FIELD

The present disclosure belongs to the technical field of biomedicine and relates to a kit for detecting an anti-proteasome subunit alpha type 1-immunoglobulin G (IgG) antibody.

### REFERENCE TO AN ELECTRONIC SEQUENCE LISTING

The contents of the electronic sequence listing (GWP20220602108.xml; Size: 2,434 bytes: and Date of Creation: Aug. 22, 2022) is herein incorporated by reference in its entirety.

### BACKGROUND ART

In recent years, there have been an increasing number of kidney diseases in children, among which autoimmune nephrotic syndrome has the highest incidence rate, seriously endangering children's physical and mental health. Autoimmune nephrotic syndrome is a type of clinical syndrome caused by increased permeability of a glomerular filtration membrane, leading to increased plasma protein filtration to generate massive proteinuria. Autoimmune nephrotic syndrome may bring symptoms to patients, mainly manifested as massive proteinuria, hypoalbuminemia, and severe edema. Ali et al. observed that after transplantation of kidneys from patients with refractory minimal change nephrotic syndrome (MCNS), the recipients had normal renal function without any proteinuria. The cause of MCNS is not all in the kidney itself but may be mainly due to the patient's internal environment. In addition, except for some pediatric patients with genetic defects, most patients with autoimmune nephrotic syndrome can improve after treatment with hormones and immunosuppressants, indirectly proving that the syndrome is closely related to the patient's autoimmunity.

For the past few years, it has been found that B-cell dysfunction also plays a vital role in autoimmune nephrotic syndrome. In recent years, multiple multicenter clinical studies worldwide have shown that rituximab (RTX) can be successfully used for treating MCNS, especially for treating refractory nephrotic syndromes with a desirable therapeutic effect. However, several studies have also found that during the treatment of hormone-dependent nephrotic syndromes with RTX, the effect of RTX in removing B cells can be maintained for approximately five months. From 6 to 7 months, the patient's condition may also relapse as the number of B cells recovers. This suggests pathological B-cell clones exist in patients with autoimmune nephrotic syndrome. Identifying and precisely removing these pathological B-cell clones is beneficial to the recovery of autoimmune nephrotic syndrome. It reduces the risk of humoral immune deficiency in patients due to indiscriminate B-cell clearance by means such as RTX. However, to date, the target antigens targeted by pathological B cells remain unclear in pediatric patients with autoimmune nephrotic syndrome. Pathologically, MCNS or focal segmental glomerulosclerosis (FSGS) is considered a podocyte disease with massive proteinuria due to loss or alteration of podocyte function. Podocytes are glomerular epithelial cells in the kidney that attach to the outside of a basement membrane of the glomerulus and are the last barrier against protein loss. Podocyte damage generally causes massive proteinuria.

The proteasome subunit is a macromolecular complex that mainly degrades proteins that are not needed by the cells or damaged. Proteasome subunit alpha type 1 is one of the proteasome families. Currently, there are more studies about proteasome subunit alpha type 1 in cancers. For example, studies have shown that proteasome subunit alpha type 1 in the proteasome pathway is closely related to controlling cell cycle progression and apoptosis. The expression of proteasome subunit alpha type 1 is significantly downregulated in hepatocellular carcinoma tumor tissues, and proteasome subunit alpha type 1 may be a potential biomarker for hepatocellular carcinoma (Jian Qin1*, et al. identification of proteasome subunit alpha type-1 as a novel biomarker in HBV-associated hepatocellular carcinoma tissue interstitial fluid by proteomic analysis. Int J Clin Exp Patho 2017; 10 (7): 7812-7820.). Yang Qian et al. reported that proteasome subunit alpha type 1 might be a potential biomarker for colon cancer (Yang Qian, Roehrl Michael H, Wang J, et al. Proteomic profiling of antibody-inducing immunogens in tumor tissue identifies proteasome subunit alpha type 1, LAP3, ANXA3, and maspin as colon cancer markers. Journal [J] Oncotarget Volume 9, Issue 3. 2018. PP 3996-4019.).

However, the expression of proteasome subunit alpha type 1 and the existence of proteasome subunit alpha type 1 autoantibodies have not been reported in nephrotic syndromes. In addition, target-based proteasome subunit alpha type 1 or its autoantibodies are not used as a serological marker in autoimmune nephrotic syndrome. There is no research on identifying autoimmune nephrotic syndrome by detecting an anti-proteasome subunit alpha type 1-IgG antibody in serum.

### SUMMARY

The present disclosure aims to provide a kit for detecting an anti-proteasome subunit alpha type 1-IgG antibody, a detection kit of target-based proteasome subunit alpha type 1, or corresponding autoantibodies thereof. The kit can detect autoantibodies from tissues (kidney biopsy) or body fluids (especially blood, plasma, and serum) by immunoreaction with the antigen protein proteasome subunit alpha type 1 (especially according to SEQ ID NO: 1).

The present disclosure provides a kit for detecting an anti-proteasome subunit alpha type 1-immunoglobulin G (IgG) antibody, including an antigen protein proteasome subunit alpha type 1, a solid phase carrier, a labeled antibody selected from the group consisting of an enzyme-labeled secondary antibody, a chemiluminescent agent-labeled secondary antibody, and a biotin-labeled secondary antibody, an antigen diluent, a sample dilution buffer, an antibody diluent, a substrate color development reagent, a washing solution, a standard, a positive quality control, and negative quality control.

The antigen protein proteasome subunit alpha type 1 has a sequence shown in SEQ ID NO: 1, as follows:

MFRNQYDNDVTVWSPQGRIHQIEYAMEAVKQGSATVGLKSKTHAVLVALK
RAQSELAAHQKKILHVDNHIGISIAGLTADARLLCNFMRQECLDSRFVFD
RPLPVSRLVSLIGSKTQIPTQRYGRRPYGVGLLIAGYDGPHIFQTCPSAN
YFDCRAMSIGARSQSARTYLERHMSEFMECNLNELVKHGLRALRETLPAE
QDLTTKNVSIGIVGKDLEFTIYDDDDVSPFLEGLEERPQRKAQPAQPADE
PAEKADEPMEH.

In the present disclosure, the antigen protein proteasome subunit alpha type 1 can be a fusion protein, using tags with certain biological or physical functions, especially an N-terminal or a C-terminal. These tags facilitate purification, immobilization, and precipitation of the antigen protein. In a preferred example, the tag may be a sequence or domain capable of specifically binding a ligand; for example, the tag peptide may be selected from the group consisting of a GST tag, a c-Myc tag, a His tag, a Flag tag, and a biotin tag.

The present disclosure indicates that the antigen protein proteasome subunit alpha type 1 may be immobilized on the solid phase carrier by physical adsorption, covalent bonding, or chemical bonding. Preferably, the solid phase carrier may be selected from the group consisting of a nitrocellulose membrane, a magnetic bead, and an enzyme-labeled microplate.

For example, the standard and the positive quality control each may be selected from the group consisting of a recombinant human anti-tag peptide IgG or a fragment thereof, and an anti-Proteasome subunit alpha type 1-IgG antibody extracted from the serum of a patient; and the negative quality control may be the serum of healthy control.

In the present study, the antigen protein proteasome subunit alpha type 1 may be expressed in bacteria such as Escherichia coli, saccharomycetes, and mammalian cells.

In the present study, the antigen protein proteasome subunit alpha type 1 may be purified by Ni column affinity chromatography, molecular sieve chromatography, ion exchange column chromatography, and hydrophobic interaction chromatography.

In the present disclosure, the biological sample may be an autoantibody-containing sample selected from the group consisting of whole blood, serum, plasma, urine, lymph, hydrothorax, and ascites, preferably mammalian (human) serum.

The kit includes a substrate color development reagent, an antigen diluent, a sample dilution buffer, an antibody diluent, and a washing solution. The substrate color development reagent may be selected from the group consisting of tetramethylbenzidine (TMB), 3-(2′-Spiroadamantane)-4-methoxy-4-(3″-phosphoryloxy) phenyl-1,2-dioxetane (AMPPD), 4-methylumbelliferyl phosphate (4-MUP), and 5-bromo-4-chloro-3-indolyl phosphate (BCIP); the antigen diluent may be a 1×PBS at pH 7.4 containing 163 mM NaCl and 1% Triton X-100; the sample dilution buffer may be a 0.01 M PBS at pH 7.4 containing 10% bovine serum albumin (BSA); the antibody diluent may be 0.01 M PBS at pH 7.4 containing 1 M D-glucose, 2% glycerol, and 0.35% Tween 20, and the washing solution may be 1×PBS at pH 7.4 containing 163 mM NaCl, 10% glycerol, and 1% Triton X-100.

In a preferred example, “immobilization” as described herein refers to binding the antigen protein proteasome subunit alpha type 1 to a water-insoluble solid phase carrier or support, preferably by covalent bonding, electrostatic interaction, hydrophobic interaction, or disulfide bond interaction, and preferably by one or more covalent bonds. The immobilization may be conducted by direct immobilization; for example, immobilized molecules are separated from an aqueous solution together with the insoluble carrier by filtration, centrifugation, or chromatography. The antigen protein proteasome subunit alpha type 1 can be reversibly or irreversibly immobilized. For example, the antigen protein is immobilized on the carrier by cleavable covalent bonds (such as disulfide bonds that can be cleaved by adding thiol-containing reagents), and this immobilization is reversible. Alternatively, if the antigen protein is immobilized to the carrier via a covalent bond that does not cleave in an aqueous solution (a bond formed by a reaction of an epoxide group with an amine group that couples a lysine side chain to an affinity column), the immobilization is irreversible. Immobilization can also be conducted indirectly, such as immobilizing an antibody with a specific affinity for the antigen protein and forming an antigen protein-antibody complex to achieve immobilization.

In the present disclosure, the antigen protein proteasome subunit alpha type 1 is immobilized by a direct enveloping method: (1) the antigen protein proteasome subunit alpha type 1 is bound to the nitrocellulose membrane or the polystyrene microplate by physical adsorption or a noncovalent bond; (2) magnetic particles with carboxyl functional groups are bound to amino groups of the antigen protein proteasome subunit alpha type 1, and the antigen protein proteasome subunit alpha type 1 is bound to the magnetic particles by chemical coupling.

In the present disclosure, the selected labeled antibody may be a horseradish peroxidase (HRP)-labeled anti-human IgG antibody, an acridinium ester-labeled anti-human IgG antibody, and a biotin-labeled anti-human IgG antibody.

The present study successfully expressed and purified the recombinant protein proteasome subunit alpha type 1 by gene recombination and prokaryotic expression. The recombinant protein is used as an antigen protein in the kit to develop a kit suitable for detecting an anti-proteasome subunit alpha type 1-IgG antibody in the serum of a patient with autoimmune nephrotic syndrome. The kit includes a qualitative or quantitative detection kit for detecting the anti-proteasome subunit alpha type 1-IgG antibody in human serum.

A kit for detecting the anti-proteasome subunit alpha type 1-IgG antibody in serum is based on indirect reaction. A proteasome subunit alpha type 1 antigen is adsorbed on the solid phase carrier as a coating antigen, the positive quality control or standard or a serum sample to be tested is added for incubation, and the labeled secondary antibody is added for reaction; if the serum to be tested contains anti-proteasome subunit alpha type 1-IgG antibody, a ternary complex of coating antigen proteasome subunit alpha type 1-anti-proteasome subunit alpha type 1-IgG antibody of serum to be tested-labeled anti-human IgG antibody is formed. The photochromogenic chemiluminescence detects light signals and fluorescence radiation methods to analyze the anti-proteasome subunit alpha type 1-IgG antibody qualitatively or quantitatively in human serum.

In the present study, the kit detected an anti-proteasome subunit alpha type 1-IgG autoantibody in some patients with autoimmune nephrotic syndrome for the first time. It was determined that a target antigen of the autoantibody is proteasome subunit alpha type 1 on podocytes. Therefore, the kit can detect the anti-proteasome subunit alpha type 1-IgG autoantibody, providing a basis for studying autoimmune nephrotic syndrome.

Compared with the prior art, the present disclosure has the following characteristics of innovation:
(1) At present, studies on proteasome subunit alpha type 1 and the anti-proteasome subunit alpha type 1-IgG antibody in patients with renal disease at home and abroad are limited to studying molecular mechanisms, and there is no quantitative detection of serum levels for these two substances in patients. The present report identifies the autoantibody against proteasome subunit alpha type 1 for the first time. A detection kit is prepared for the anti-proteasome subunit alpha type 1-IgG autoantibody, thus filling the gap in related fields at home and abroad.
(2) In the present disclosure, the kit involves qualitative analysis of the anti-proteasome subunit alpha type 1-IgG antibody in human serum, where solid-phase membrane immunoassay is simple in operation, with less reagent consumption nearly ten times lower than traditional ELISA; in addition, the nitrocellulose membrane (NC) membrane has an extremely strong adsorption capacity of close to 100%, such that trace antigen can be completely absorbed and immobilized on the NC membrane; the NC membrane adsorbed with antigen or antibody or with existing results can be stored for a long time (at −20° C. for half a year) without affecting the activity. In addition, the kit for the qualitative detection of the anti-proteasome subunit alpha type 1-IgG antibody in human serum by the solid-phase membrane immunoassay introduces a biotin-avidin amplification system, thereby significantly improving the detection sensitivity.
(3) In the present disclosure, a kit for quantitatively detecting the anti-proteasome subunit alpha type 1-IgG antibody in human serum is related to magnetic particle-based chemiluminescence immunoassay uses magnetic particles as a solid phase carrier with a diameter of only 1.0 μm. This dramatically increases the coating surface area, increases the amount of antigen adsorbed, improves the reaction rate, and makes cleaning and separation easier, thereby reducing contamination and the probability of cross-infection. On the other hand, the anti-human IgG is directly labeled by an acridinium ester luminescent agent with a simple, rapid, and catalyst-free chemical reaction; the acridinium ester has flash-type chemiluminescence and can achieve a maximum emission intensity after 0.4 s by starting a luminescent reagent (H₂O₂, NaOH); the acridinium ester has a half-life of 0.9 s, which can be terminated within 2 s, thereby realizing rapid detection.

### BRIEF DESCRIPTION OF THE DRAWINGS

FIG. 1 shows that proteasome subunit alpha type 1 on podocytes is a target antigen for autoantibodies in a patient with autoimmune nephrotic syndrome; FIG. 1A: a primary antibody is a two-dimensional electrophoresis protein spot of healthy human serum; FIG. 1B: a primary antibody is a two-dimensional electrophoresis protein spot in the serum of a patient with autoimmune nephrotic syndrome; FIG. 1C: mass spectrometry identification of the target antigen proteasome subunit alpha type 1;

FIG. 2 shows SDS-PAGE identification of the expressed recombinant protein proteasome subunit alpha type 1;

FIG. 3 shows the detection of an anti-proteasome subunit alpha type 1-IgG antibody in the serum of a patient with autoimmune nephrotic syndrome by a solid-phase membrane immunoassay kit.

FIG. 4 shows a schematic diagram for detecting the anti-proteasome subunit alpha type 1-IgG antibody by a magnetic particle-based chemiluminescence immunoassay kit.

FIG. 5 shows a schematic diagram of the antigen protein proteasome subunit alpha type 1 coated with carboxyl magnetic particles.

FIG. 6 shows the detection of the anti-proteasome subunit alpha type 1-IgG antibody in patients with various types of renal diseases, where NS: autoimmune nephrotic syndrome, HSP: Henoch-schonlein purpura, HSPN: Henoch-schonlein purpura nephritis, IgAN: IgA nephropathy, and NC: healthy children.

FIG. 7 shows a ROC curve to evaluate the value of the anti-proteasome subunit alpha type 1-IgG antibody as a serological marker for the diagnosis of patients with autoimmune nephrotic syndrome.

### DETAILED DESCRIPTION OF THE EMBODIMENTS

The present disclosure will be described in further detail below with reference to the accompanying drawings and specific embodiments. The following embodiments are only intended to illustrate the disclosure rather than limiting the scope of the disclosure.

### Example 1 Proteasome Subunit Alpha Type 1 on Podocytes as a Target Antigen for Autoantibodies in a Patient with Autoimmune Nephrotic Syndrome

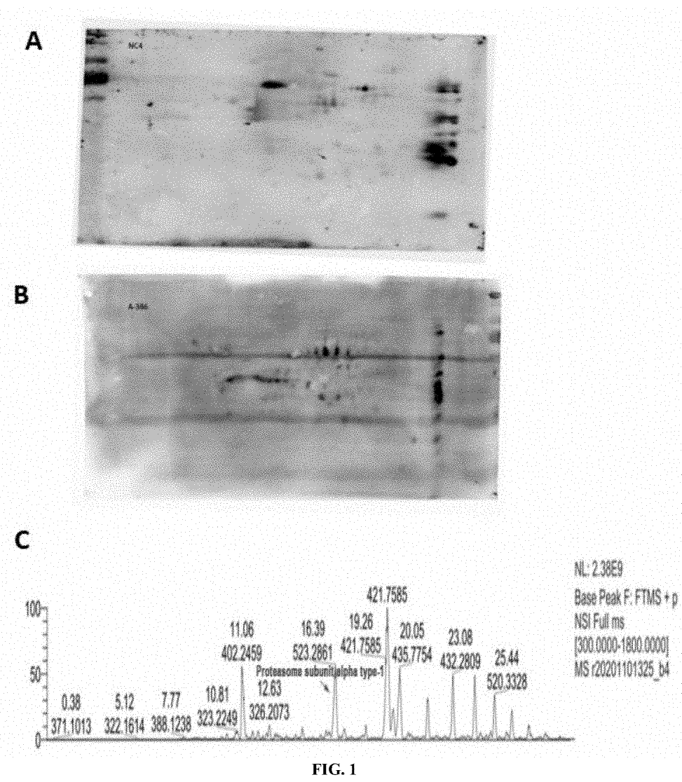

In the present disclosure, through many clinical and molecular mechanism studies in an early stage, it was found for the first time that patients with nephrotic syndrome have a higher serum IgG level. Furthermore, it was confirmed that proteasome subunit alpha type 1 on podocytes was the target antigen for autoantibodies in patients with autoimmune nephrotic syndrome. Therefore, detecting the presence and quantitative levels of the anti-proteasome subunit alpha type 1-IgG antibody in serum was helpful for the early identification of autoimmune nephrotic syndrome, especially for screening patients with related symptoms. The specific implementation included the following: (1) extraction of total protein from glomerular podocytes: a sample of a podocyte line (MPCS) was cultured, washed 2 to 3 times with PBS, and subjected to extensive lysis on ice using a focused sonicator (Covaris S220, Gene) in lysis buffer containing 30 mM Tris-HC1, 8 M urea, 4% CHAPS and a protease inhibitor (#ab65621; Abcam, 1:200 dilution), and the sample was centrifuged at 12,000 g and 4° C. for 30 min. The supernatant was collected to obtain the total protein of glomerular podocytes. The total protein concentration of the glomerular podocytes was determined using a BCA protein concentration assay kit. (2) Two-dimensional electrophoresis: the total protein of glomerular podocytes was subjected to two-dimensional electrophoresis and transferred to a nitrocellulose membrane; sera of healthy people and patients with autoimmune nephrotic syndrome were used as primary antibodies for incubation separately, and secondary antibody was added for development, as shown in FIG. 1A and FIG. 1B. (3)

Matrix-assisted laser desorption ionization time-of-flight mass spectrometry (MALDI-TOF-MS): after development in step (2), differential analysis was conducted on positive spots; protein spots were strongly positive in nephrotic syndrome patients, and negative or weakly positive in healthy people on the two-dimensional electrophoresis gel were selected, and the selected protein spots were cut out from the gel; a dried gel was digested with trypsin (0.1 μg/μl ), 10 μl of 25 mM ammonium bicarbonate was added to a reaction mixture, incubated overnight at 37° C., and peptides were extracted from the gel with trifluoroacetic acid (0.1%). The extracted peptide was analyzed by a MALDI-TOF-MS mass spectrometer to obtain a mass spectrum of the peptide, which was identified as proteasome subunit alpha type 1 protein, as shown in FIG. 1C.

### Example 2 Expression and Purification of the Recombinant Antigen Protein Proteasome Subunit Alpha Type 1

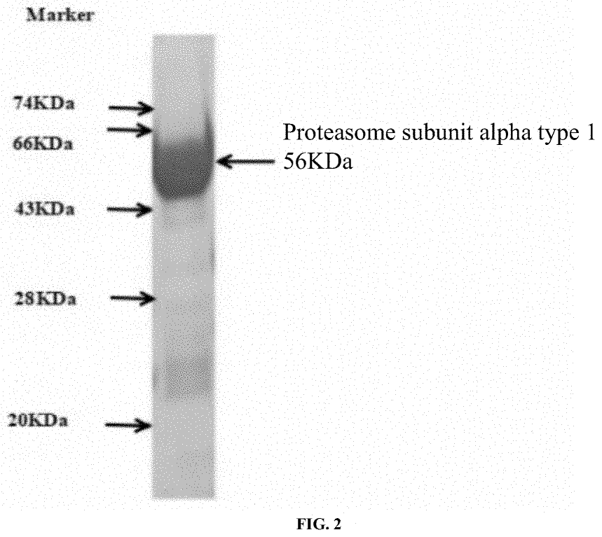

A gene encoding the proteasome subunit alpha type 1 protein was used as a template for PCR amplification by genetic engineering, and an expression carrier was constructed for protein expression. Tag peptides containing GST tags were on expressed antigen proteins. The expressed recombinant protein was purified by nickel column affinity chromatography, ion affinity chromatography, hydrophobic interaction chromatography, and molecular sieves, and the molecular weight of the recombinant protein proteasome subunit alpha type 1 was identified by SDS—PAGE as 56 KDa, as shown in FIG. 2.

### Example 3 Optimization of the Reaction Conditions of a Kit by Orthogonal Experimental Design

According to a coating concentration of the antigen proteasome subunit alpha type 1 (50 μg/ml, 90 μg/ml, 120 μg/ml, and 150 μg/ml), each reaction time (15 min, 30 min, and 45 min), temperature (25° C. and 37° C.), and an optimal dilution of enzyme-labeled secondary antibody (1:100, 1:500, 1:1000, 1:1500), an orthogonal table was selected, each factor was repeated at two levels to determine standard positive serum and standard negative serum, and a ratio (P/N) of the highest light signal value (P) of the positive serum and the lowest light signal value (N) of the negative serum was selected. By the orthogonal design, the kit had an optimal antigen Proteasome subunit alpha type 1 coating concentration of 90 μg/ml, and the solid-phase membrane immunoassay for anti-Proteasome subunit alpha type 1-IgG antibody kit had an optimal antigen-antibody reaction temperature of 25° C., an optimal antigen-antibody reaction time of 30 min, and an optimal working dilution of the optimal labeled anti-human IgG antibody at 1:1000; the magnetic particle-based chemiluminescence immunoassay for anti-Proteasome subunit alpha type 1-IgG antibody kit had an optimal antigen-antibody reaction temperature of 37° C., an optimal antigen-antibody reaction time of 15 min, and an optimal working dilution of the optimal labeled anti-human IgG antibody at 1:1000.

### Example 4 Preparation of a Solid-Phase Membrane Immunoassay Kit for Detecting an Anti-Proteasome Subunit Alpha Type 1-IgG Antibody

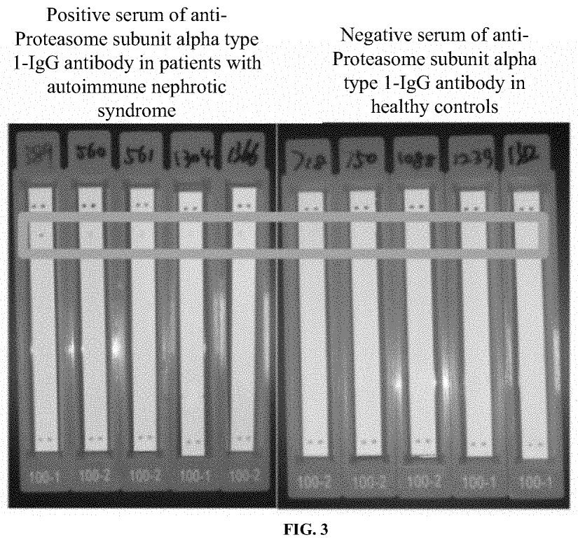

4.1. Composition of a solid-phase membrane immunoassay kit for detection of the anti-Proteasome subunit alpha type 1-IgG antibody:
1. Antigen: recombinant protein proteasome subunit alpha type 1
2. Solid phase carrier: Satourius CN140 nitrocellulose membrane
3. Positive quality control (standard): human anti-GST tag IgG (purchased from Huzhou Yingchuang)
4. Negative quality control: serum of healthy controls
5. Labeled antibody: biotin-labeled anti-human IgG antibody
6. Antigen diluent
7. Sample dilution buffer
8. Antibody diluent
9. Washing solution
10. Enzyme working solution: alkaline phosphatase-streptavidin
11. Substrate color development reagent: BCIP color development reagent.
4.2. The detection steps of the solid-phase membrane immunoassay kit for the detection of the anti-proteasome subunit alpha type 1-IgG antibody included the following:
4.2.1. coating and blocking: 8 μl of protease subunit alpha type 1 antigen at a concentration of 90 μg/ml was added dropwise directly to the nitrocellulose membrane and dried in a 37° C. incubator for 30 min, and the nitrocellulose membrane was blocked on a detection plate with 200 μl of 5% BSA in a 37° C. incubator for 30 min. After discarding the blocking solution, the nitrocellulose membrane was washed twice with a washing solution.
4.2.2. Antigen incubation: 10 μl of an antibody standard or serum to be tested diluted with diluent was added to the detection plate, while negative and positive controls were set up at the same time and then incubated at 25° C. for 30 min, where three parallel wells were set for each sample.
4.2.3. Secondary antibody incubation: The liquid in the detection plate was discarded, the plate was washed five times with a washing solution for 1 min each time, and 20 μl of a 1:1000 biotin-labeled anti-human IgG antibody was added and incubated at 25° C. for 30 min.
4.2.4 Color development: The liquid in the detection plate was discarded, the plate was washed five times with the washing solution for 1 min each time, and 500 μl of alkaline phosphatase-streptavidin was added and incubated at room temperature for 20 min. The liquid in the detection plate was discarded, and the plate was washed five times with the washing solution for 1 min each time. A color development reagent BCIP was added and reacted at room temperature for 20 min, and the detection plate was rinsed with running water to stop the enzyme reaction. The nitrocellulose membrane test strip was removed and dried with a hairdryer and qualitatively determined with a colorimetric card by the naked eye. Those with obvious brown spots were positive, as shown in FIG. 3. Alternatively, the membrane strip was scanned on a developing device, and analysis software that comes with the developing device used the reference standard concentration as ordinate, and the gray value read by the device as an abscissa to draw a standard curve for the semiquantitative analysis of anti-Proteasome subunit alpha type 1-IgG antibody levels in serum.

### Example 5 Preparation of a Magnetic Particle-Based Chemiluminescence Immunoassay Kit for Detecting an Anti-Proteasome Subunit Alpha Type 1-IgG Antibody

5.1. Composition of a magnetic particle-based chemiluminescence immunoassay kit for detection of the anti-Proteasome subunit alpha type 1-IgG antibody:
1. Antigen: recombinant protein proteasome subunit alpha type 1
2. Solid phase carrier: magnetic particles with carboxyl functional groups
3. Positive quality control (standard): human anti-GST tag IgG (purchased from Huzhou Yingchuang)
4. Negative quality control: serum of healthy controls
5. Labeled antibody: acridinium ester-labeled anti-human IgG antibody
6. Antigen diluent
7. Sample dilution buffer
8. Antibody diluent
9. Washing solution
10. Preexcitation solution: H₂O₂
11. Excitation solution: NaOH
5.2. Principle of a magnetic particle-based chemiluminescence immunoassay kit for detection of the anti-Proteasome subunit alpha type 1-IgG antibody

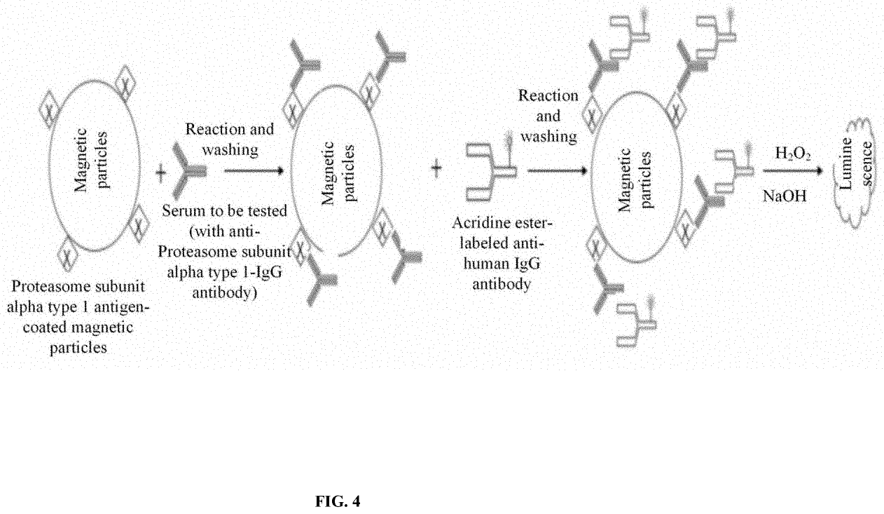

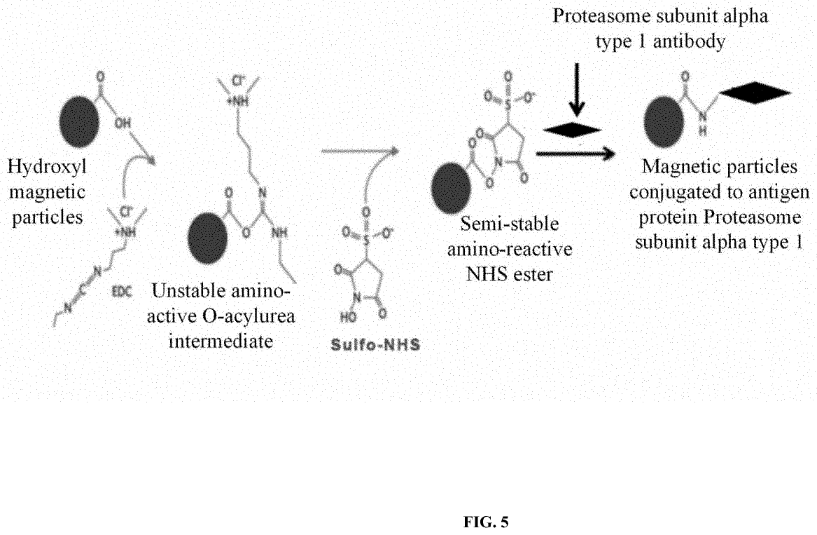

The chemiluminescence immunoassay kit is an analytical method combining magnetic separation, immunoassay, and chemiluminescence. The kit used an indirect method to quantitatively detect anti-Proteasome subunit alpha type 1-IgG antibody in human serum: a magnetic microparticle fluid was mixed with a diluted sample, and specific anti-Proteasome subunit alpha type 1-IgG antibodies were bound to the Proteasome subunit alpha type 1 antigen-coated magnetic particles; after washing, an acridinium ester-labeled anti-human IgG antibody was added to form “a Proteasome subunit alpha type 1 antigen-anti-Proteasome subunit alpha type 1-IgG antibody-acridinium ester-labeled anti-human IgG antibody complex”. Under the action of an external magnetic field, the unbound substance and the complex formed by the immune reaction were separated, the supernatant was discarded, a precipitated complex was washed, and the preexcitation solution (H₂O₂) and the excitation solution (NaOH) were added to conduct a luminescence reaction. Under alkaline conditions, the acridinium ester molecule was attacked by hydrogen peroxide to generate dioxyethane, which was unstable and decomposed into CO₂ and N-methylacridone in an electronically excited state. When returning to the ground state, N-methylacridone emitted light with a wavelength of 430 nm, and the luminescence intensity was determined using a chemil. The concentration of anti-proteasome subunit alpha type 1-IgG antibody was proportional to the luminescence intensity, and the concentration of anti-proteasome subunit alpha type 1-IgG antibody in the serum to be tested was calculated by a calibration curve, as shown in FIG. 4.
5.3. Preparation of proteasome subunit alpha type 1 antigen-coated magnetic particles
5.3.1 The principle of coating magnetic particles with proteasome subunit alpha type 1 antigen is as follows: the carboxyl functional groups contained on the surface of the magnetic particles react with an EDC (1-(3-dimethylaminopropyl)-3-ethylcarbodiimide) solution to generate an unstable amino-active O-acylurea intermediate; the intermediate reacts with NHS (N-hydroxysuccinimide) to form a semistable amino-reactive NHS ester; the semistable amino-reactive NHS ester reacts with amino groups on the antigen protein proteasome subunit alpha type 1 to form proteasome subunit alpha type 1 antigen-coated magnetic particles, as shown in FIG. 5.
5.3.2. For the EDC/NHS activation of the carboxyl magnetic particles, the specific steps were as follows:
a) A total of 10 mg of magnetic particles was washed three times with 20 mM MES and separated by a magnet, and the supernatant was discarded.
b) The washed magnetic particles were resuspended in 100 μl of 20 mM MES to a final 100 mg/ml concentration.
c) Fifty microliters of 20 mg/ml EDC and 50 μl of 24 mg/ml Sulfo-NHS that were prepared in PBS were added to the cleaned magnetic particles in sequence, mixed well, and allowed to stand at room temperature for activation for 30 min.
d) After the action of an external magnetic field, the supernatant was discarded, and the magnetic particles were washed with 400 μl of 0.05 M PBS, diluted with 400 μl of a preservation solution, and stored for later use.
5.3.3 Crosslinking of activated magnetic particles with antigen protein Proteasome subunit alpha type 1: precooled 1 ml of 20 mM MES was added to wash the activated magnetic particle solution twice; 200 p1 of the 2 mg/ml antigen protein Proteasome subunit alpha type 1 was added to the activated magnetic particles, mixed thoroughly, and allowed to stand for reaction at room temperature for 16 h; after the reaction, PBS containing 0.2% Tween 20 at pH 7.4 was added, and the magnetic particles were washed twice; PBS containing 0.2% Tween 20 and 0.2% BSA at pH 7.4 was added until a final concentration of the magnetic particles was 10 mg/ml, mixed well, and allowed to stand for reaction at room temperature for 30 min; after the reaction, a supernatant was discarded, and the magnetic particles were resuspended in PBS containing 0.2% Tween 20, 0.2% BSA at pH 7.4, such that the cross-linking of the activated magnetic particles and the antigen protein Proteasome subunit alpha type 1 was completed.
5.4. To prepare the acridinium ester-labeled anti-human IgG antibody, the specific steps were as follows:
a) A 2 mg/mL acridinium ester solution was prepared by dimethylformamide.
b) A 1 mg/mL anti-human IgG antibody was prepared by a 0.2 M (pH 8.0) carbonate buffer.
c) The acridinium ester and the anti-human IgG antibody was thoroughly mixed at a molar ratio of 4:1 and reacted for 40 min.
d) The reaction was terminated by 20 μl of carbonate buffer containing 5% lysine.
e) desalting and impurity removal were conducted to obtain an acridinium ester-labeled anti-human IgG antibody solution with higher purity.
5.5. The steps of detecting anti-proteasome subunit alpha type 1-IgG antibody in serum by a magnetic particle-based chemiluminescence immunoassay kit were as follows:
5.5.1 Serum to be tested: 100 μl of a diluted serum to be tested or an anti-GST-tagged IgG standard was added to 100 μl of the magnetic particle solution coated with the antigen protein proteasome subunit alpha type 1 and reacted at 37° C. for 15 min, while negative and positive controls were set up.
5.5.2 Labeled antibody: A total of 400 μl of a washing solution was added to wash three times for 1 min each time, and 100 μl of an acridinium ester-labeled anti-human IgG antibody diluted by 1:1000 was added and reacted at 37° C. for 15 min.
5.5.3 Signal detection: After discarding the supernatant, the precipitated complexes were washed with 400 μl of the washing solution three times for 1 min each time and 100 μl of a preexcitation solution (H₂O₂) and 100 μl of an excitation solution (NaOH) were added for the reaction. The luminescence signal was detected by a chemiluminometer, and the luminescence value was recorded. The concentration of anti-proteasome subunit alpha type 1-IgG antibody was proportional to the luminescence intensity in the serum to be tested, and the concentration of anti-proteasome subunit alpha type 1-IgG antibody in the serum to be tested was calculated by a standard curve.

### Example 6 Clinical Use of a Kit for Detecting an Anti-Proteasome Subunit Alpha Type 1-IgG Antibody in Serum

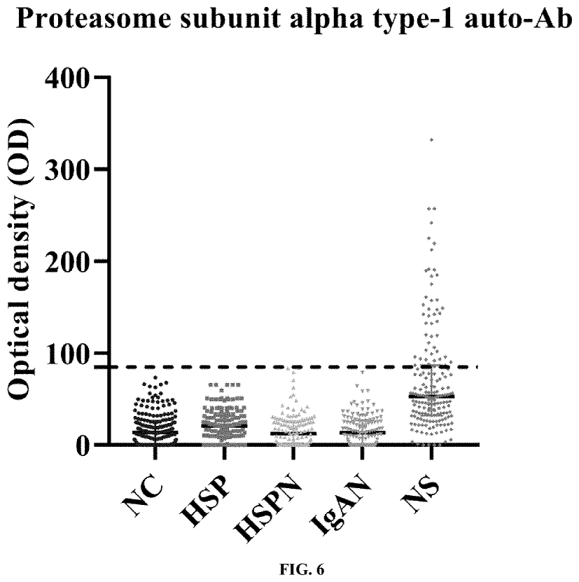

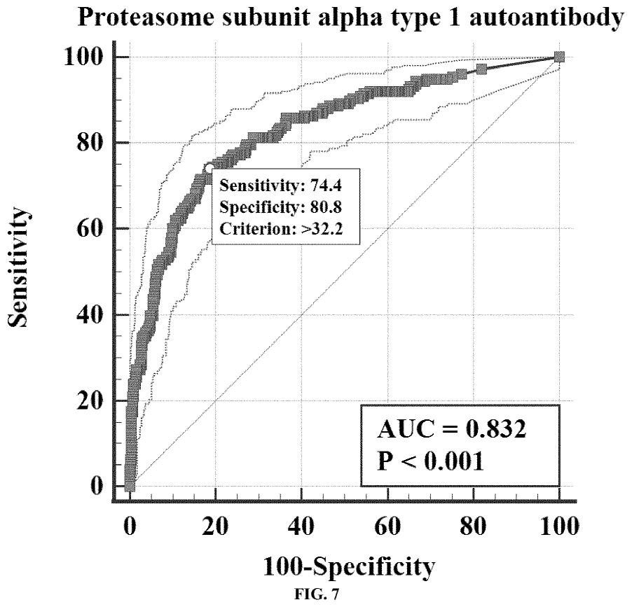

6.1 Subjects included patients diagnosed with various types of nephropathies from June 2018 to June 2020, including 466 cases of NS, 168 cases of HSP, 137 cases of HSPN, 133 cases of IgAN, and 195 cases of NC during the same period. Serum samples were obtained from various nephropathy patients and healthy controls. All subjects had their first serum sample collection before immunosuppressive therapy.
6.2 Detection of anti-Proteasome subunit alpha type 1-IgG antibodies in patients with various types of nephropathies: The kit for the present disclosure was used to detect anti-Proteasome subunit alpha type 1-IgG antibody levels in the serum of patients diagnosed with various types of nephropathies from June 2018 to June 2020, including 466 cases of NS, 168 cases of HSP, 137 cases of HSPN, 133 cases of IgAN, and 195 cases of NC during the same period. The results showed positive anti-proteasome subunit alpha type 1-IgG antibodies in patients with autoimmune nephrotic syndrome and negative anti-proteasome subunit alpha type 1-IgG antibodies in patients with HSPN, HSP, IgAN, and NC, as shown in FIG. 6.
6.3 ROC curve: The value of anti-proteasome subunit alpha type 1-IgG antibody was evaluated as a serological marker in the diagnosis of patients with autoimmune nephrotic syndrome; the detection results of anti-proteasome subunit alpha type 1-IgG antibody in patients with autoimmune nephrotic syndrome in 6.2 were analyzed using the ROC curve to evaluate the value of the anti-proteasome subunit alpha type 1-IgG antibody in the diagnosis of autoimmune nephrotic syndrome. The results showed that the anti-proteasome subunit alpha type 1-IgG antibody was a desirable serological marker for the diagnosis of patients with autoimmune nephrotic syndrome; the anti-proteasome subunit alpha type 1-IgG antibody (with a cutoff value greater than 32.2 as a criterion) as a serological marker for the diagnosis of the autoimmune nephrotic syndrome had a sensitivity of 74.4%, a specificity of 80.8% and an area under the curve of 0.832, as shown in FIG. 7.

1. Use of an antigen protein proteasome subunit alpha type 1 in preparation of a kit for detecting autoimmune nephrotic syndrome, wherein the kit comprises an antigen protein proteasome subunit alpha type 1, a solid phase carrier, a labeled antibody, an antigen diluent, a sample dilution buffer, an antibody diluent, a substrate color development reagent, a washing solution, a standard, a positive quality control, and a negative quality control; the antigen protein proteasome subunit alpha type 1 has a sequence shown in SEQ ID NO: 1; the labeled antibody is an enzyme-labeled secondary antibody; the antigen protein proteasome subunit alpha type 1 has a tag peptide; the standard and the positive quality control each are an anti-proteasome subunit alpha type 1-IgG antibody extracted from serum, and the negative quality control is the serum of a healthy control; and the autoimmune nephrotic syndrome is diagnosed by detecting the anti-proteasome subunit alpha type 1-IgG antibody in a serum sample of a patient.

2. (canceled)

3. (canceled)

4. The use according to claim 1, wherein the tag peptide is a His tag.

5. The use according to claim 1, wherein the antigen protein proteasome subunit alpha type 1 is purified by nickel column affinity chromatography.

6. (canceled)

7. The use according to claim 1, wherein the antigen protein proteasome subunit alpha type 1 is immobilized on a nitrocellulose membrane solid phase carrier.

8. The use according to claim 7, wherein the antigen protein proteasome subunit alpha type 1 is directly immobilized on the solid phase carrier by physical adsorption.

9. The use according to claim 1, wherein the substrate color development reagent is tetramethylbenzidine (TMB); the antigen diluent is a 1×PBS at a pH value of 7.4 containing 163 mM NaCl and 1% Triton X-100; the sample dilution buffer is a 0.01 M PBS at a pH value of 7.4 containing 10% bovine serum albumin (BSA); the antibody diluent is the 0.01 M PBS at a pH value of 7.4 containing 1M D-glucose, 2% glycerol, and 0.35% Tween 20; and the washing solution is the 1×PBS at a pH value of 7.4 containing the 163 mM NaCl, 10% glycerol, and the 1% Triton X-100.
